(19) United States
(12) Patent Application Publication
(10) Pub. No.: US 2005/0279365 A1
(43) Pub. Date: Dec. 22, 2005

(54) Anti-snoring apparatus and method

(76) Inventors: Dennis F. Armijo, Albuquerque, NM (US); Mohsen Shahinpoor, Albuquerque, NM (US)

Correspondence Address: DENNIS F ARMIJO, 6300 MONTANO RD. NW, SUITE D, ALBUQUERQUE, NM, 87120, US

(21) Appl. No.: 10/872,181

(22) Filed: Jun. 18, 2004

(51) Int. Cl. A62B 18/02; A61F 5/56

(52) U.S. Cl. 128/848

(57) ABSTRACT

An anti-snoring piercing in the form of specially designed thin mesh-like symmetric structure resembling a pair of wings connected symmetrically by means of a middle bar-like bridge. A middle bar-like bridge is mounted under the uvula while the two symmetric wing parts are placed over the soft palate and pierced on the soft palate in the mouth to provide stiffness to uvula-soft palate assembly and thus prevent snoring. A stiffening member is attached to the uvula by means of at least one piercing post with a backing or with an adhesive. Each of these embodiments stiffens the uvula and prevents snoring.

### BACKGROUND OF THE INVENTION

1. Field of the Invention (Technical Field)

This invention is generally in the field of anti-snoring devices and methods. More specifically, it is in the field of devices and methods that inhibit fluttering of the soft palate during sleep, as air flows past it.

2. Background Art

There are many devices, methods, drugs, and surgical procedures available to treat snoring during sleep. In addition to a large variety of mechanical devices that basically control the tongue movement during sleep and are about the size of a mouthpiece of the type worn by athletes engaged in contact sports, there are also devices that work directly on the uvula or the soft palate. Typical patents are, for example, U.S. Pat. No. 6,626,181 to Knudsen, et al., entitled “stiff snoring implant” issued September 2003, which discusses extensively and makes extensive references to stiffening implants which are used to prevent the soft palate from fluttering during sleep. In another U.S. Pat. No. 6,467,485 to Schmidt, entitled “anti-snoring device and method”, issued October 2002, discusses an apparatus that inhibits snoring by having a handle and a distal free end that is disposed at an angle relative to the handle to facilitate placing the distal free end against the soft palate at the back of a throat. A flexible material such as a sheet of paper is placed into overlying relation to the distal free end and a layer of adhesive is disposed in overlying relation to the paper. When the distal free end of the apparatus is pressed against the soft palate, the adhesive quickly adheres to the soft pallet so that the paper adheres to the soft pallet when the apparatus is withdrawn. The paper supposedly is to provide stiffness to the palate to inhibit fluttering vibration of the soft palate. The commonly used surgical procedures include:
1. Injection snoreplasty in which a biocompatible material such as Sotradecol, a sclerosing agent typically used to treat varicose veins, is injected into the soft palate (SP) to create scar tissues and to stiffen SP and prevent fluttering
2. Laser-assisted uvula palatoplasty (LAUP) in which a laser is used to ablate (vaporize) the uvula and portions of the soft palate
3. Uvulopalatopharyngoplasty (UPPP) in which the palate is folded over on itself and stitched into place thus surgically trimming and tightening the throat tissue and creating stiffening scar tissues.

These surgical procedures are rather drastic, painful, and expensive and do have their own risks.

The mouthpiece devices are also inconvenient and hard to wear and maintain during sleep. Drugs and medications have also been ineffective in solving the snoring problem. What is needed is a user-friendly non-drug and non-surgical alternative to cure snoring. Thus, the present invention offers an anti-snoring device that is affixed to the soft palate or uvula by piercing. The apparatus is in the form of specially designed thin mesh-like symmetric structure resembling a pair of wings connected symmetrically by means of a middle bar-like bridge. The middle bar-like bridge is mounted under the uvula while the two symmetric wing parts are placed over the soft palate and pierced on the soft palate in the mouth to provide stiffness to uvula-soft palate assembly and thus prevent snoring. In another embodiment the apparatus is also affixed to the soft palate or uvula by piercing and keeps the uvula from “fluttering” by the stiffening apparatus. The stiffening member could also be attached by an adhesive.

### SUMMARY OF THE INVENTION (DISCLOSURE OF THE INVENTION)

This invention is generally related to a prosthesis that inhibits fluttering of the soft palate during sleep, as air flows past it. The general structure of the proposed anti-snoring device is that it is a user-friendly easily mountable anti-snoring piercing in the form of a light weight thin bio-compatible, such as a plastic mesh structure with a special geometry resembling a pair of wings to fit over the soft palate with piercing anchors such that the middle part of the pair of wings is placed under the uvula and the wings are placed over the soft palate in a symmetric configuration to create stiffening reinforcement to prevent the soft palate from fluttering due to breathing during sleep and thus prevent snoring. An alternative embodiment is a single stiffening structure with a single or double piercing with a backing on the piercing element to constrict the movement of the uvula and soft palate. Yet another embodiment comprises a stiffening member is attached to the soft palate and uvula with an adhesive.

An important object of this invention is to significantly advance the art of anti-snoring devices and methods by providing a prosthesis that is pierced or affixed over the soft palate to inhibit snoring.

Another object of the said invention is to provide a methodology where snoring is inhibited by a mesh-like light weight bio-compatible material structure that is pierced by means of piercing anchors or pins over the soft palate.

Another important object of the invention is to provide a user-friendly system that inhibits fluttering of the soft palate.

Other objects, advantages and novel features, and further scope of applicability of the present invention will be set forth in part in the detailed description to follow, taken in conjunction with the accompanying drawings, and in part will become apparent to those skilled in the art upon examination of the following, or may be learned by practice of the invention. The objects and advantages of the invention may be realized and attained by means of the instrumentalities and combinations particularly pointed out in the appended claims.

### BRIEF DESCRIPTION OF THE DRAWINGS

The accompanying drawings, which are incorporated into and form a part of the specification, illustrate several embodiments of the present invention and, together with the description, serve to explain the principles of the invention. The drawings are only for the purpose of illustrating a preferred embodiment of the invention and are not to be construed as limiting the invention. In the drawings:

FIG. 1 shows a first embodiment of the anti-snoring apparatus.

FIG. 2 is a view of the anti-snoring device of FIG. 1 mounted over the soft palate and under the uvula in the mouth by means of piercing.

FIG. 3 is a side view of a human head showing where the prosthesis of FIG. 1 for inhibiting fluttering of the soft palate is placed FIGS. 4a and 4b shows a second embodiment of the invention with a pierced stiffening member.

FIGS. 5a and 5b shows the embodiment of FIGS. 4a and 4b with a differently configured stiffening member.

FIG. 6 shows a third embodiment of the invention showing the stiffening member affixed to the uvula with an adhesive.

### DESCRIPTION OF THE PREFERRED EMBODIMENTS (BEST MODES FOR CARRYING OUT THE INVENTION)

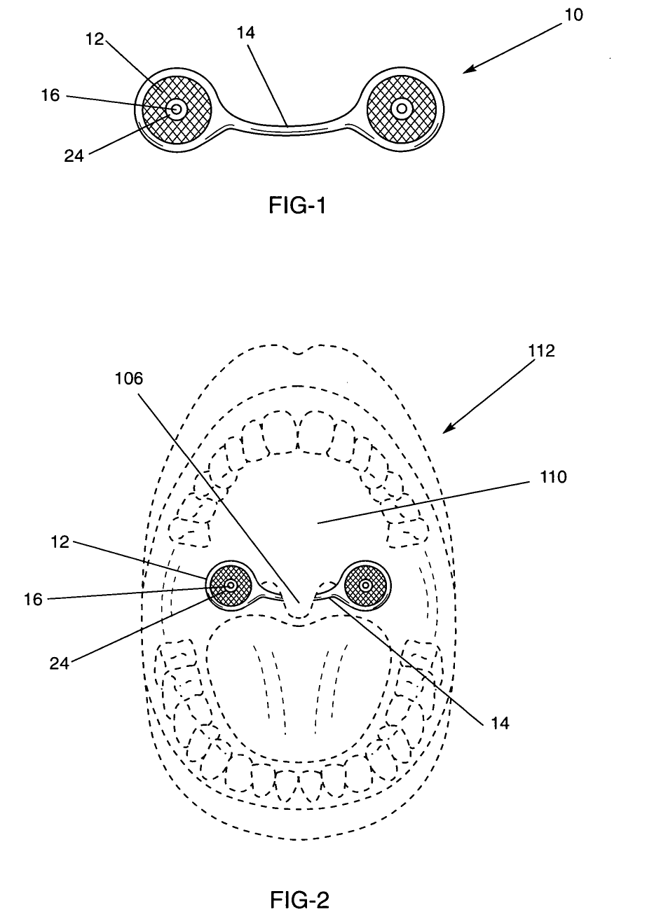

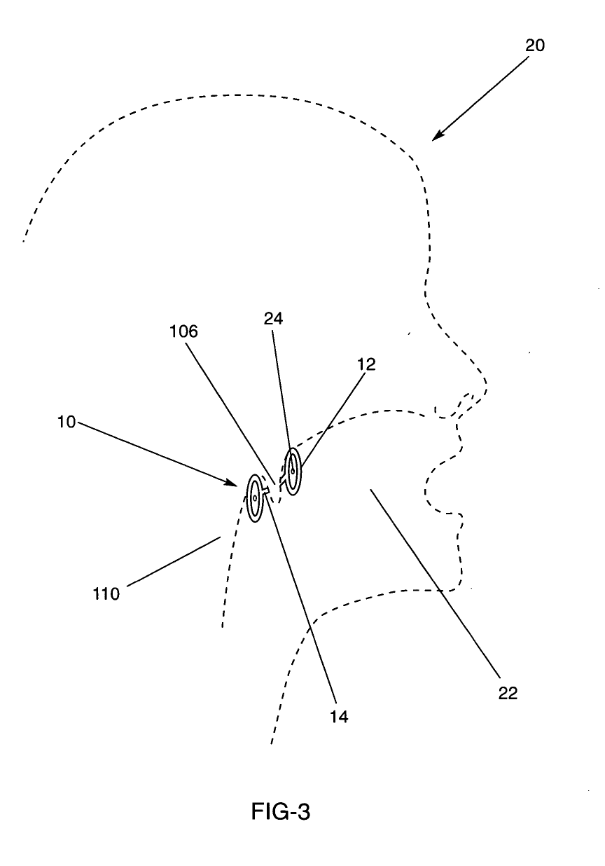

The present invention is a stiffening member that is affixed to the uvula 106 and/or soft palate 110 to prevent “fluttering” of the same and thus, to keep a person from snoring. FIG. 1 illustrates a first embodiment of the anti-snoring apparatus 10, of the present invention. The preferred apparatus resembles a pair of wings 12 connected to each other by means of a bar-like bridge 14. The preferred wings 12 are constructed from a mesh-like material for rigidity and are light-weight. The preferred apparatus 10 has piercing posts 16, one for each wing 12, and backings or plugs 24 and are connected in the middle by the bar-like bridge section 14. In order to install the anti-snoring apparatus 10 in a mouth, the individual could lie down on his or her back with his or her mouth open for a professional piercer to install the anti-snoring apparatus 10 in a way similar to the known process of uvula piercing. Referring to FIG. 2, which shows how the middle part of the structure or the bridge 14 is placed under the uvula 106 in a mouth 112. Wings 12 are placed over the soft palate 110 in a symmetric manner. To secure wings 12 on the soft palate 110, piercing posts 16 are used to pierce through the soft palate 110 and backing or plugs 24 are used to secure the piercing posts 16 in place, as shown in FIG. 2. FIG. 3 depicts a side view of a human head 20 and mouth 22 and how the anti-snoring apparatus 10 is placed under the uvula 106 and over the soft palate 110. Bridge 14 is placed under the uvula 106, and thus keep uvula 106 from drooping back while a person is sleep on his or her back. Furthermore, the placement of the light weight wings 12 on the soft palate 110 by means of piercing posts 16 stiffens the soft palate 110 and prevent its fluttering during sleep and thus prevent snoring.

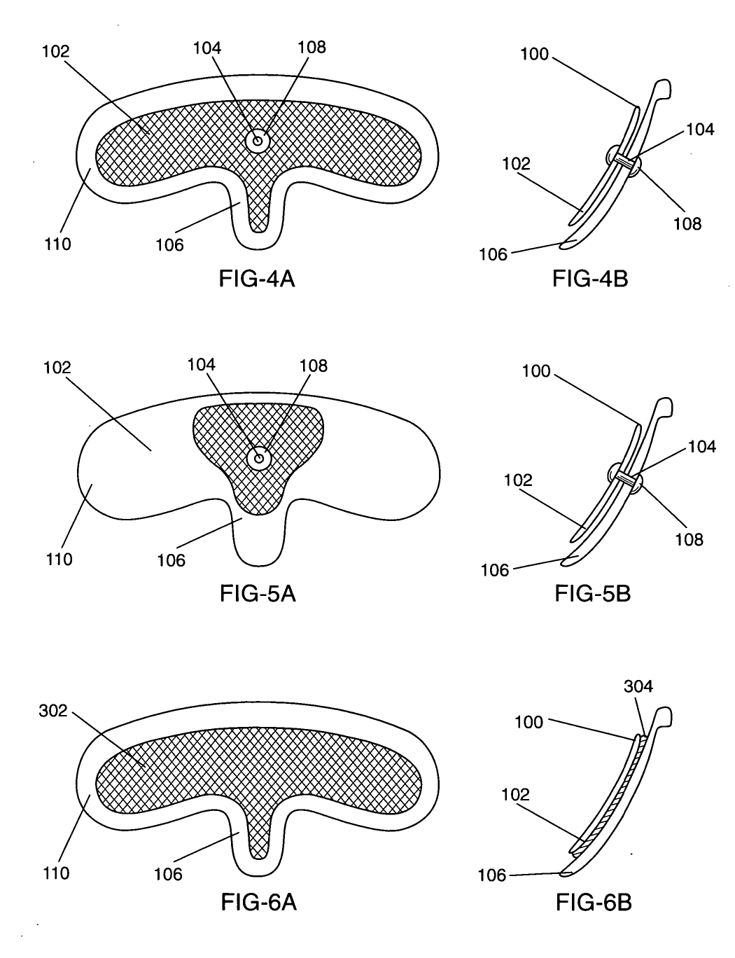

A second embodiment of the present invention is shown on FIGS. 4a, 4b, 5a and 5b. In the embodiment of FIGS. 4a and 4b, the anti-snoring apparatus 100 comprises of a stiffening member 102 containing a piercing post 104 which is attached to the uvula 106 or near the uvula on the soft palate 110, as shown. The piercing post 104 has a backing or plug 108 to keep anti-snoring apparatus 100 in place. The backing 108 is secured to the uvula 106 or soft palate 110 with adequate pressure, as shown in FIG. 4b, to force the stiffening member 102 against the uvula 106 and soft palate 110 thus, keeping the uvula 106 from fluttering during a person's sleep. Stiffening member 102 is preferably a rigid light-weight material that will not be affected by moisture and will not degrade. It can be a mesh-like material, above or can be constructed from plastic, stainless steel or other well known materials. Stiffening member 102 can be elongated as shown in FIG. 4a to effectively cover the uvula 106. A second configuration of the anti-snoring apparatus 200 is shown in FIGS. 5a and 5b. In this configuration, uvula 106, is partially covered by stiffening member 202. In addition, a portion of the soft palate 110 is compressed onto stiffening member 202. The stiffening members 102, 202 could contain additional piercing posts (not shown) for providing additional coverage and compression force of the soft palate 110 and uvula 106 against the stiffening member 102, 202. There are several factors to consider in optimizing the anti-snoring apparatus 100, 200 in general and for each individual use. The main focus is on adequately covering the uvula 106 and/or the soft palate 110 with a stiffening member 102, 202, adequately providing enough pressing force of the stiffening member 102, 202 against the uvula 106 and/or soft palate 110 and configuring the backing or plug 108 to provide a pressing force and a sufficient surface area of the backing or plug 108 to keep fluttering of the uvula 106 and/or soft palate 110, as previously described. Thus, the number of piercing posts 104, the configuration of the stiffening member 102, 202, the amount of pressing force on the stiffening member 102, 202 and the configuration of the backing or plug 108 can be optimized to provide an effective anti-snoring apparatus. With a single piercing post 104, it would be preferable, but not necessarily required, to have a symmetrical stiffening member. In addition, a single piercing would be minimally intrusive on a person. The size of the stiffening member 102, 202 can be optimized for each user by the size of the user's uvula, by the piercer trying different sizes of stiffening members until the fluttering ceases. For aesthetic purposes, the anti-snoring apparatus can be made from materials in a similar color as the uvula or soft palate, or could be decorated in a variety of ways to emphasize and show the piercing. Thus, a user can attempt to hide the piercing or to show it off. As previously described, the anti-snoring apparatus can be installed similarly as the first described embodiment, which is well known in the piercing art.

A third embodiment of the invention is show on FIG. 6. This embodiment involves the same concept of affixing a stiffening member 302 to the uvula 106 and/or soft palate 110, however instead of affixing the stiffening member by piercing, it is affixed by an adhesive 304. This method is less intrusive than piercing, however, stiffening member 302 would have to be reapplied or a new stiffening member attached as the adhesive 304 breaks down. There are several adhesives available, such as dental adhesives, that are well known in the art. The stiffening member could also be clipped to the uvula or soft palate by bending members attached to the edge of the stiffening member and are bent around and over the uvula and soft palate in order to maintain contact between the uvula and soft palate to the stiffening member (not shown). Additionally, spring loaded clamping devices can be disposed on the stiffening member to clamp the stiffening member to the uvula or soft palate (not shown). The configuration of the stiffening members can be as described in the previous embodiments.

Although the invention has been described in detail with particular reference to these preferred embodiments, other embodiments can achieve the same results. Variations and modifications of the present invention will be obvious to those skilled in the art and it is intended to cover in the appended claims all such modifications and equivalents. The entire disclosures of all references, applications, patents, and publications cited above, are hereby incorporated by reference.

1. An apparatus for treating snoring caused by fluttering of the uvula and soft palate, the apparatus comprising:
a stiffening member configured to be affixed and substantially adjoined to a predetermined portion of the uvula and soft palate;
at least on piercing post for affixing and adjoining said stiffening member; and
a backing for each piercing post from the at least two piercing posts for keeping said stiffening member in place and for providing a predetermined pressing force of said stiffening member to the uvula and the soft palate.

2. The apparatus of claim 1 wherein said stiffening member comprises a mesh-like material.

3. The apparatus of claim 1 wherein said stiffening member comprises a bio-compatible material.

4. The apparatus of claim 1 wherein said stiffening member is configured to conform to a shape of the predetermined portion.

5. An apparatus for treating snoring caused by fluttering of the uvula and soft palate, the apparatus comprising:
a pair of wing like structures configured to be affixed to the soft palate by a first piercing post affixed to a first wing and a piercing post affixed to a second wing from said pair of wing like structures;
a first backing removably affixed to the first piercing post and a second backing removably affixed to the second piercing post for keeping said wing like structures in place; and
a bar bridge affixed at a first end to said first wing and a second end affixed to said second wing, wherein said apparatus is mounted so said bar bridge is positioned substantially under the uvula.

6. The apparatus of claim 5 wherein said pair of wing like structures comprise a mesh-like symmetric structure.

7. The apparatus of claim 5 wherein said apparatus comprises a biocompatible material.

8. The apparatus of claim 5 wherein said biocompatible material comprises a member from the group of biocompatible medical grade plastics and biocompatible medical grade metals.

9. An apparatus for treating snoring caused by fluttering of the uvula and soft palate, the apparatus comprising:
a stiffening member configured to be affixed and substantially adjoined to a predetermined portion of the uvula and soft palate; and
an adhesive disposed on said stiffening member for affixing and substantially adjoining said stiffening member.

10. The invention of claim 9 wherein said stiffening member comprises a mesh-like material.

11. The invention of claim 9 wherein said stiffening member comprises a biocompatible material.

12. The apparatus of claim 11 wherein said biocompatible material comprises a member from the group of biocompatible medical grade plastics and biocompatible medical grade metals.

13. The invention of claim 9 wherein said stiffening member is configured to conform to a shape of the predetermined portion.

14. A method for treating snoring caused by fluttering of the uvula and soft palate, the method comprising the steps of:
a) providing a stiffening member configured to be affixed to a predetermined portion of the uvula and the soft palate; and
b) affixing and adjoining the stiffening member to the predetermined portion of the uvula and the soft palate.

15. The method of claim 14 wherein the step of affixing and adjoining comprises piercing the stiffening member to the uvula or soft palate.
